(19) United States
(12) Patent Application Publication
(10) Pub. No.: US 2004/0064851 A1
(43) Pub. Date: Apr. 1, 2004

(54) Transgenic plants which produce isomalt

(76) Inventors: Markwart Kunz, Worms (DE); Ralf Mattes, Stuttgart (DE); Mohammed Munir, Kindenheim (DE); Manfred Vogel, Neuleiningen (DE)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/380,529

(22) PCT Filed: Jul. 12, 2001
(86) PCT No.: PCT/EP01/08055

(30) Foreign Application Priority Data
Sep. 20, 2000 (DE) 100 47 286.9

(51) Int. Cl. A01H 1/00; C12N 15/82; C12P 19/04

(52) U.S. Cl. 800/284; 435/101

(57) ABSTRACT

The present invention concerns a transgenic plant which can produce isomaltulose, a transgenic plant which can produce 6-O-α-D-glucopyranosyl-D-sorbitol, a transgenic plant which can produce 1-O-α-D-glucopyranosyl-D-mannitol, a transgenic plant which can produce a mixture of 1,6-GPs and 1,1-GPM, propagation and harvest material from these plants, and a process for producing these transgenic plants.

### FIELD OF THE INVENTION

The present invention concerns a transgenic plant which can produce isomaltulose, a transgenic plant which can produce 6-O-α-D-glucopyranosyl-D-sorbitol (called 1,6-GPS in the following), a transgenic plant which can produce 1-O-α-D-glucopyranosyl-D-mannitol (called 1,1-GPM in the following), a transgenic plant which can produce a mixture of 1,6-GPS and 1,1-GPM, propagation and harvest material from these plants, and processes for producing these transgenic plants.

### BACKGROUND OF THE INVENTION

Sucrose isomerases which isomerize the glycosidic bond between the monosaccharide units of sucrose and so can catalyze the conversion of sucrose to isomaltulose and trehalulose are known from DE 44 14 185 C1 (e.g., from the microorganisms Protaminobacter rubrum and Erwinia rhapontici). This document describes the DNA sequences which code for sucrose isomerase, and cells transformed by it.

Processes are also known for producing Palatinit® (also called isomalt, or hydrogenated isomaltulose), a nearly equimolar mixture of 1,6-GPS and 1,1-GPM, as well as the individual components, 1,1-GPM and 1,6-GPS, from sucrose. The processes carry out an enzymatic conversion of sucrose to isomaltulose and then a chemical hydrogenation of the isomaltulose produced to give the two stereoisomers, 1,6-GPS and 1,1-GPM. For instance, Schiweck (alimenta 19 (1980), 5-16) published a process for getting Palatinit® which involves enzymatic conversion of sucrose to isomaltulose and subsequent hydrogenation of the isolated isomaltulose on Raney nickel catalysts. The conversion of sucrose to isomaltulose is accomplished with the microorganism Protaminobacter rubrum. The isomaltulose obtained in that way is converted to 1,6-GPS and 1,1-GPM by hydrogenation in the presence of Raney nickel catalysts, after which it is concentrated by evaporation and cooling crystallization processes.

EP 0 625 578 B1 describes processes for obtaining sugar alcohol mixtures containing 1,1-GPM and 1,6-GPS, in which sucrose is first converted enzymatically into a mixture containing isomaltulose and trehalulose. The product so obtained is hydrogenated catalytically to a mixture containing 1,1-GPM, 1,6-GPS and 1-O-α-D-glucopyranosyl-D-sorbitol (1,1-GPS).

DE 195 23 008 A1 discloses a process for producing mixtures of 1,1-GPM and 1,6-GPS. It involves hydrogenation of isomaltulose using catalysts containing ruthenium, nickel, or mixtures of them at pressures of less than 50 atmospheres.

DE 197 01 439 A1 discloses processes for hydrogenating isomaltulose by means of a carrier-bound nickel catalyst. The processes produce mixtures of 1,6-GPS and 1,1-GPM.

DE 197 05 664 A1 discloses processes for producing mixtures of hydrogenated isomaltulose enriched with 1,6-GPS or 1,1-GPM. One process described in that document involves production of mixtures enriched with 1,6-GPS and/or 1,1-GPM from hydrogenated isomaltulose or from mixtures containing hydrogenated isomaltulose. When this process is used, 1,6-GPS can be produced in pure form by concentrating a mother liquor enriched with 1,6-GPS under specified conditions and cooling crystallization.

A sorbitol dehydrogenase from a microorganism of the genus Gluconobacter is known from the German patent application DE 199 63 126.3. With it, isomaltulose can be converted directly to 1,6-GPS.

Finally, a mannitol dehydrogenase has been isolated form a microorganism of the genus Pseudomonas (Brünker et al., Biochemica et Biophysica Acta, ¹³⁵¹ (1997), 157-167). It can convert isomaltulose to 1,1-GPM.

The processes at the state of the art are considered disadvantageous with respect to producing Palatinit®, its individual components, and its precursors, primarily for the following reasons.

First, nearly all the processes for producing the specified substance require that sucrose first be isolated by physical-chemical processes from sugar beets, for instance, and purified for the following steps of the process. Second, then, further complex processes follow in the continued processing of sucrose to 1,6-GPS and/or 1,1-GPM. Various physical, chemical and/or biological processes must be used in different reactors. In order to get 1,6-GPS directly from sucrose, for example, at least two separate enzymatic conversions are required. As a general rule, costly purification steps must be carried out in the course of those conversions. Much the same applies to production of 1,1-GPM and isomalt. Among other things, for instance, getting 1,1-GPM from sucrose requires carrying out at least one enzymatic conversion, one chemical hydrogenation using catalysts, special hydrogenation reactors and industrial hydrogen, as well as subsequent separations to isolate 1,1-GPM from the previously obtained mixture of 1,1-GPM and 1,6-GPS.

Therefore to carry out several process steps at substantial technological cost is a substantial disadvantage of the process known at the state of the art. Furthermore, because the products so obtained are intended for use in the food industry, the processes must be selected so that no toxic substances, such as from the catalysts, get into the final product. That requires more steps of purification which often result in yields of the final product not being satisfactory.

Thus the technological problem on which the present invention is based is to provide a simple, economical and selective recovery of isomaltulose and its hydrogenation products, especially 1,6-GPS, from 1,1-GPM or from mixtures containing it.

### BRIEF SUMMARY OF THE INVENTION

The present invention solves this technical problem by producing transgenic plants, especially transgenic potatoes or transgenic sugar beets, which can produce isomaltulose in at least one of their cells from sucrose produced in the plant. In particular, the invention concerns a plant previously outlined which has not only the ability to produce isomaltulose from the sucrose which it produces, but also to produce 1,6-GPS and/or 1,1-GPM from that. Such a plant provides, in a surprising and advantageous manner, an in vivo system for producing Palatinit®, its individual components, and the precursor, isomaltulose, which allows direct production of the desired final products at the site of origin of the starting material, sucrose. Expensive isolation, purification, and/or hydrogenation processes are avoided here. Furthermore, no toxic substances of any sort are used.

The invention further solves the problem on which it is based by providing processes for producing the specified plants.

### BRIEF DESCRIPTION OF THE DRAWINGS

The invention is explained in more detail with the following figures and examples. The figures show:

FIG. 1 A restriction map of the plasmid pHWG279.1, a HindIII fragment about 1.7 kb in size, which contains the sequence (smuA*) coding for sucrose isomerase in the vector pBR322.

FIG. 2 A restriction map of the plasmid pHWG469, which contains the native gene for sorbitol dehydrogenase (sdh) from Gluconobacter suboxidans in the vector pBR322.

### DETAILED DESCRIPTION OF THE INVENTION

One preferred embodiment of the present invention, therefore, concerns a transgenic plant, especially a transgenic sugar beet or potato which is able to produce isomaltulose from sucrose in at least one of its cells. Such a plant is a valuable raw material for production of Palatinit® which can, for example, be obtained from the plant by chemical hydrogenation after isolation of the isomaltulose. Obviously, one can also produce mixtures in which the ratio of 1,1-GPM to 1,6-GPS differs from a 1:1 ratio of 1,1-GPM to 1,6-GPS. Such a plant can also be made the starting point for further genetic manipulation leading finally to production of a complete metabolic pathway from sucrose to Palatinit® or its individual components.

In connection with this invention, a transgenic plant from which isomaltulose can be produced from sucrose formed in the plant is understood to be a plant which contains a stable integrated nucleotide sequence which can be expressed in it, which codes for activity of a sucrose isomerase. A sucrose isomerase catalyzes isomerization of sucrose to isomaltulose, in the process of which the α1→β2 glycosidic bond between glucose and fructose in the sucrose is converted into a different glycosidic bond, specifically into an α1→β6 bond. According to the invention, suitable nucleotide sequences which code for the activity of a sucrose isomerase are known from, among others, microorganisms of the general Protaminobacter, Erwinia, Serratia, Leuconostoc, Pseudomonas, Agrobacterium or Klebsiella. DE 44 14 185 C1 discloses isolation and cloning of nucleotide sequences coding for sucrose isomerase from the microorganisms Protaminobacter rubrum and Erwinia rhapontici. That document discloses completely the present teaching with respect to description and production of the DNA sequences, and protection is requested for these DNA sequences in the context of the invention.

One particularly preferred embodiment of the present invention concerns a transgenic plant, especially a transgenic sugar beet or potato which can, in at least one of its cells, produce isomaltulose from sucrose and which can generate 1,6-GPS from the isomaltulose so produced.

In connection with the invention, a transgenic plant which can produce 1,6-GPS from the isomaltulose formed is understood to be a plant which contains a stable nucleotide sequence integrated into it and expressible in it which codes for the activity of a sucrose isomerase, and thus can produce isomaltulose from sucrose, and which also contains a stable integrated and expressible nucleotide sequence which codes for the activity of a sorbitol dehydrogenase. A sorbitol dehydrogenase activity specifically reduces isomaltulose to 1,6-GPS. The German patent application DE 199 63 126.3 discloses a sorbitol dehydrogenase from the microorganism Gluconobacter suboxidans which is suitable according to the invention. The specific document discloses completely the present teaching with respect to description and production of the DNA sequence, and protection is requested for this DNA sequence in the context of the invention.

Another particularly preferred embodiment of the present invention concerns a transgenic plant, especially a transgenic sugar beet or potato which can, in at least one of its cells, produce isomaltulose from sucrose produced in the plant, and which can generate 1,1-GPM from the isomaltulose so produced.

In connection with the invention, a transgenic plant which can generate 1,1-GPM from the isomaltulose formed is understood to be a plant which contains a stable nucleotide sequence integrated into it and expressible in it which codes for the activity of a sucrose isomerase and so can produce isomaltulose from sucrose, and which also contains a stable integrated and expressible nucleotide sequence which codes for the activity of a mannitol dehydrogenase. A mannitol dehydrogenase activity reduces isomaltulose specifically to 1,1-GPM. Brünker et al. describe, in Biochimica et Biophysica Acta, 1351 (1997), 157-167, a mannitol dehydrogenase suitable according to the invention from the microorganism Pseudomonas fluorescens DSM 50106. The specific document discloses completely the present teaching with respect to description and production of the DNA sequence, and protection is requested for this DNA sequence in the context of the invention.

Another particularly preferred embodiment of the invention concerns a transgenic plant, especially a transgenic sugar beet or potato, which can, in at least one of its cells, generate isomaltulose from the sucrose formed in the plant, and can generate 1,6-GPS and 1,1-GPM from the isomaltulose so generated, in, for instance, a 1:1 mixture.

In connection with the invention, a transgenic plant which can generate 1,6-GPS and 1,1-GPM from the isomaltulose formed is understood to be a plant which contains a stable nucleotide sequence integrated into it and expressible in it which codes for the activity of a sucrose isomerase and so can produce isomaltulose from sucrose, and which also contains either a stable integrated and expressible nucleotide sequence which codes for the activity of a sorbitol dehydrogenase, and contains a stable integrated and expressible nucleotide sequence which codes for the activity of a mannitol dehydrogenase, or a stable integrated and expressible nucleotide sequence which codes for the activity of an unspecifically hydrogenating polyol dehydrogenase.

Another embodiment of the present invention concerns a transgenic plant, especially a sugar beet or a potato, which contains in at least one of its cells a stable integrated and expressible nucleotide sequence which codes for the activity of a sorbitol dehydrogenase.

In another embodiment, the present invention produces a transgenic plant, especially a sugar beet or potato, which contains in at least one of its cells a stable integrated and expressible nucleotide sequence which codes for the activity of a mannitol dehydrogenase. The two plants named above are advantageous to the extent that they allow preparation of the enzymes sorbitol dehydrogenase and mannitol dehydrogenase. Furthermore, the plants named can serve as starting materials for production of transgenic plants which produce Palatinit® from sucrose, in which nucleotide sequences coding for sucrose isomerase must be introduced into those plants.

The transgenic plants can be plants of quite different species, genera, families, orders and classes. That is, they can be either monocotyledenous or dicotyledenous plants, such as algae, moss, ferns or gymnosperms. Transgenic plants can also include calluses, plant cell cultures, and parts, organs, tissues, harvests or propagation materials from them.

The invention particularly provides that the transgenic plant is a useful plant, especially a useful plant which can produce sucrose in its storage system, such as sugar cane or sugar beets. The invention likewise concerns propagation materials and harvest products of the plants according to the invention, such as flowers, fruits, storage organs, beet roots, stems, seeds, tubers, roots, leaves, rootstocks, seedlings, cuttings, etc.

In connection with the present invention, the expression “in at least one of its cells” means that a transgenic plant contains at least one cell, though preferably a multiplicity of cells, containing one or more stable integrated nucleotide sequences which code for the activity of a sucrose isomerase and/or the activity of a sorbitol dehydrogenase and/or the activity of a mannitol dehydrogenase. The cells are preferably cells in which sucrose is produced or stored. In the case of a transgenic sugar beet, the cells are preferably cells of the sugar beet storage organ, i. e., root cells, while in the case of a transgenic potato they are preferably cells of the tuber.

The nucleotide sequence can preferably be integrated into the cell nucleus, but it may also be integrated into the plastid genome or into the mitochondrial genome, preferably so that it is inherited stably in the next generation.

The present invention is also concerned with transgenic cells which contain the nucleotide sequences named above, and transgenic plants derived from such cells.

Such cells can be distinguished from naturally occurring cells by the fact that they each contain one or more of the coding nucleotide sequences named above, which do not occur naturally in those cells, or that the coding nucleotide sequences named above are integrated at a locus in the genome at which they do not occur naturally, or that the coding nucleotide sequences named above appear in other than the natural number of copies. In addition, the plants described above are distinguished by the metabolic activities and the expression of the named enzymes produced according to the invention. The invention produces such plants in an advantageous manner such that their vigor, phenotype and/or culture conditions are entirely the same as those of a wild type plant.

In connection with the present invention, the expression “stable integrated and expressible nucleotide sequence” means that a nucleotide sequence is linked to nucleic acid elements which provide stable integration of that nucleotide sequence into the genome of a plant so that the integrated nucleotide sequence is replicated along with the genome components of the plant cell that are present naturally, and is also linked with regulatory DNA elements which assure transcription of the nucleotide sequence and subsequent expression of the product coded by the nucleotide sequence.

In the preferred embodiment, the coding regions of these nucleotide sequences are linked with regulatory elements for expression of the nucleotide sequence previously specified in plant cells, especially in sense orientation. The regulatory elements include, in particular, promoters which assure transcription in the plant cells. Essentially both homologous and heterologous promoters can be considered for expression of the previously specified nucleotide sequences. They may be promoters which cause a constitutive expression, or promoters which are active only in a specific tissue, at a specific time during plant development, or only at a time determined by external influences. Furthermore, the nucleotide sequences specified above are, in the preferred embodiment, linked with a termination sequence which causes correct ending of the transcription and addition of a poly-A tail to the transcript. Such elements are described in the literature (Gielen et al., EMBO J., 8 (1989), 23-29).

In one preferred embodiment of the present invention, expression of the nucleotide sequences coding for the enzymatic activities is achieved by the fact that these nucleotide sequences are expressed in at least one plant cell under the control of tissue-specific or organ-specific, and especially storage-organ-specific promoters.

One example of tissue-specific promoters for expression of the nucleotide sequences coding for enzymatic activities is the vicilin promoter from Pisum sativum (Newbigin et al., Planta, 180 (1990), 461-470). In another preferred embodiment, the invention provides for use of, for example, the Arabidopsis promoter AtAAP1 (expression in endosperm and during early embryonic development) or AtAAP2 (expression in phloem of the funiculus) (Hirner at al., Plant J., 14 (1988), 535-544) for expression of the previously specified nucleotide sequences in the epidermis and parenchyma of so-called “sink organs”.

A particularly preferred embodiment provides expression of the nucleotide sequences coding for the enzymatic activities in the plant organs which store large quantities of sucrose. Those include, for instance, the root of the sugar beet, the stem of sugar cane, or the tubers of the AGPase-antisense Line 93 of the potato, the “sucrose potato” (Müller-Röber et al., Mol. Gen. Genet., 224 (1990), 136-146). Expression of the nucleotide sequences coding for the enzymatic activities can, for instance, be achieved by using the B33 promoter of the B33 gene from potatoes (Rocha-Sosa et al., EMBO J., 8 (1988), 23-29).

In another embodiment of the invention, it is possible to provide for use of constitutively expressing promoters such as the CaMV 35S promoter, the companion-cell-specific rolC promoter from Agrobacterium, or the enhanced PMA4 promoter (Morian et al., Plant J., 19 (1999), 31-41).

In another embodiment, the invention concerns transgenic plants in which nucleotide sequences in the reading frame coding for the enzymatic activities are fused to a signal sequence which codes for a signal peptide for uptake of the gene products exhibiting the enzymatic activities into the endoplasmic reticulum of a eucaryotic cell. Therefore the invention provides that the nucleotide sequences can be given signal sequences which allow localization of the gene products in specific compartments of the cell. In particular, for example, one can consider signal sequences coding for signal peptides which cause uptake of proteins into the endoplasmic reticulum. That can be demonstrated by the fact that they are detectable as the precursor proteins but not as processed mature proteins. As is well known, the signal peptides are removed proteolytically during uptake into the endoplasmic reticulum. In one embodiment of the invention, for instance, it can be provided that a signal peptide such as the shortened N-terminal sequence of the proteinase inhibitor PI II from potato (Keil et al., Nucl. Acids Res., 14, (1986), 5641-5650; Schaewen et al., EMBO Journal, 9 (1990), 3033-3044) is used. That results in uptake of the gene product into the endoplasmic reticulum with subsequent secretion into the apoplastic space. Obviously, other signal sequences can also be used according to the invention.

In another embodiment, the invention provides that the nucleotide sequences coding for the enzymatic activities are fused to a signal sequence which codes for a signal peptide for uptake into the endoplasmic reticulum of an eucaryotic cell, especially a plant cell, and for further transfer into the vacuole. Vacuolar localization of the gene product is particularly advantageous. For example, according to the invention, one can use signal peptides for vacuolar localization of lectin from barley (Raikhel and Lerner, Dev. Genet., 12 (1991), 255-260), signal sequences coding for 43 amino acids in the amino-terminal region of the ripe phytohemaglutinin of the bean (Tague et al., Plant Cell, 2, (1990), 533-546), and signal sequences from a patatine gene of potato.

It is particularly preferred according to the invention to utilize a signal sequence from the patatine B33 gene which codes for the 23 amino-terminal amino acids of the propeptide to localize the gene product in the vacuole (Rosahl et al., Mol. Gen. Genet., 203 (1986), 214-220), i.e., nucleotides 736 to 804. This sequence can be obtained both as a fragment from the genomic DNA of the potato and from the cDNA of the B33 gene. Fusion of the extended B33 signal sequence with the coding nucleotide sequence results in uptake of their gene products in the vacuole.

In another particularly preferred embodiment, it is provided that the nucleotide sequences which code for the enzymatic activities are not fused with a signal sequence, so that the expressed gene products remain in the cytosol.

The invention also concerns processes for producing the specified transgenic plants, including transformation of one or more plant cells with a vector, especially a plasmid containing one or more nucleotide sequences selected from the group consisting of a nucleotide sequence coding for the activity of a sucrose isomerase, a nucleotide sequence coding for the activity of a sorbitol dehydrogenase, and a nucleotide sequence coding for the activity of a mannitol dehydrogenase, integration of the coding nucleotide sequences contained in this vector or plasmid into the genome of the transformed cell(s), optionally including its/their signal sequences and/or regulatory elements, and regeneration of the plant cell(s) to intact fertile transformed plants which produce sorbitol dehydrogenase, mannitol dehydrogenase and/or sucrose isomerase.

Numerous processes are available to introduce DNA into a plant host cell. In many processes it is necessary that the nucleotide sequences to be introduced occur in cloning and/or expression vectors. Vectors are essentially plasmids, cosmids, viruses, bacteriophages, shuttle vectors, and other vectors commonly used in genetic engineering. Vectors can have other functional units which stabilize the vector in a host organism and/or make its replication possible. Vectors can also contain regulatory elements functionally linked to the nucleotide sequence obtained and which allow expression of the nucleotide sequence in a host organism. Such regulatory units can be promoters, enhancers, operators and/or transcription termination signals. Vectors also frequently contain marker genes which allow selection of the host organisms containing them, such as antibiotic resistance genes.

Processes for introducing DNA into plant cells include transformation of plant cells with T-DNA using Agrobacterium tumefaciens or Agrobacterium rhizogenes as transforming agents, protoplast fusion, microinjection, electroporation of DNA, introduction of DNA by means of biolistic methods and other possibilities. The processes of microinjection and electroporation of DNA into plant cells do not themselves place any special requirements on the plasmids to be used. Simple plasmids can be used, such as pUC derivatives. However, if whole plants are to be regenerated from cells transformed in that manner, a selectable marker should be present.

Depending on the process used to introduce coding nucleotide sequences into the plant cells, it may be necessary for the vector to contain other DNA sequences. For example, if the Ti or R1 plasmid is used to transform plant cells, it is necessary for at least the right border sequence, and often both the right and left border sequences of the Ti and R1 plasmid cells to be linked as flank regions with the genes being introduced. When Agrobacterium is used for transformation, the DNA being introduced must be cloned in special plasmids, in either an intermediary vector or a binary vector. Because of sequences homologous with sequences in the T-DNA, intermediary vectors can be integrated into the Ti or R1 plasmid of Agrobacterium through homologous recombination. Those also contain the vir region required for transfer of the T-DNA. Intermediary vectors cannot replicate in agrobacteria. The intermediary vector can be transferred to Agrobacterium tumefaciens by means of a helper plasmid. In contrast, binary vectors can replicate both in agrobacteria and in E. coli. They contain a gene for a selection marker and a linker or polylinker framed by the right and left T-DNA border regions. Binary vectors can be transformed directly into agrobacteria (Holsters et al., Mol. Gen. Genet., 163 (1978), 181-187). The Agrobacterium which serves as the host cell should contain a plasmid carrying a vir region. This vir region is necessary for the transfer of the T-DNA into the plant cell. The Agrobacterium transformed in that way is used to transform plant cells. Use of T-DNA to transform plant cells is described in the following publications, among others: EP-A-120 516; Hoekema: The Binary Plant Vector System, Offsetdrukkerej Kanters. B. V., Alblasserdam (1985), Chapter V; Fralej et al., Crit. Rev. Plant. Sci., 4, 1-46, and An et al., EMBO J., 4 (1985), 277-287). Plant explants can be co-cultivated with Agrobacterium tumefaciens or Agrobacterium rhizogenes to transfer DNA into the plant cells. The whole plants can be regenerated from the infected plant material, such as leaf fragments, stem segments, roots, protoplasts, or plant cells cultivated in suspension, in a suitable medium which contains antibiotics or biocides to select transformed cells. One preferred process for transformation of beet cells using Agrobacterium tumefaciens is disclosed in EP 0 517 833 B1.

Other possibilities for introducing foreign DNA using the biolistic process or by means of protoplast transformation are disclosed in, for example, Willmitzer, L., Transgenic Plants, In: Biotechnology, A Multi-Volume Comprehensive Treatise (H. J. Rehm, G. Reed., A. Pühler, P. Stadler, Eds.), Volume 2 (1993), 627-659, VCH Weinheim, New York, Basel, Cambridge. Alternative systems for transforming monocotyledenous plants include electrically or chemically induced uptake of DNA into protoplasts, electroporation of partially permeabilized cells, macroinjection of DNA into inflorescences, microinjection of DNA into microspores and pro-embryos, DNA uptake by germinating pollen, and DNA uptake in embryos by soaking (Potrykos, Physiol. Plant (1990), 269-273). More recent studies indicate that monocotyledenous plants can also be transformed using vectors based on Agrobacterium (Chan et al., Plant Mol. Biol., 22 (1993), 491-506; Hiei et al., Plant J., 6 (1994) 271-282; Bytebier et al., Proc. Natl. Acad Sci. USA, 84 (1987), 5345-5349; Raineri et al., Bio/Technology, 8 (1990), 33-38; Gould et al., Plant. Physiol., 95 (1991), 426-434; Mooney et al., Plant, Cell Tiss. & Org. Cult., 25 (1991), 209-218; Li et al., Plant Mol. Biol. 20 (1992), 1037-1048). Some of the transformation systems mentioned above have become established for various species of cereals. Examples are the electroporation of tissues, transformation of protoplasts and DNA transfer by particle bombardment in regenerable tissues and cells (Jähne et al., Euphytica, 85 (1995), 35-44). Transformation of wheat has been described by Maheshwari et al., Critical Reviews in Plant Science, 14 (2) (1995), 149-178; and transformation of maize has been described by Brettschneider et al., Theor. Appl. Genet., 94 (1997), 737-748, and by Ishida et al., Nature Biotechnology, 14 (1996), 745-750.

### EXAMPLE 1

### Production of Vectors which Contain a Nucleotide Sequence Coding for Sucrose Isomerase

A series of constructs was produced which contained, in a binary vector, each of a promoter expressible in plants, the nucleotide sequence from Protaminobacter rubrum coding for sucrose isomerase, and the polyadenylation signal of T-DNA octopine synthase (Gielen et al., 1984). The coding nucleotide sequence was either fused with the signal sequence of the patatine gene of the potato (Rosahl et al., Mol. Gen. Genet., 203 (1986), 214-220), which causes vacuolar localization of the gene product, or it was used without a vacuolar target sequence to achieve expression in the cytosol of the particular plant cell. Both the CaMV 35 S-promoter and the promoter of the patatine gene B33 of the potato (Rocha-Sosa et al., EMBO J., 8 (1989), 23-29) were used as promoters with which organ-specific expression in potato tubers and in the storage root of the sugar beet could be achieved. In the case of the potato, the binary vector pBinB33-Hyg (Becker, Nucl. Acids Res., 18 (1990), 203) was used. It already contains the B33 promoter and the polyadenylation signal, as well as the Hyg resistance gene as a marker. In the case of the sugar beet, the binary vector pGA492 (An, Plant Physiol., 81 (1986), 86-91) was used. It has a kanamycin resistance gene. Agrobacteria were transformed with the plasmids obtained. The transformed agrobacteria were used to transform either potato or sugar beet. The structure of the plasmid UL8-19 is described in the following. The sequence coding for sucrose isomerase at the 5′ end is fused “in frame” with that for the signal peptide of the patatine gene and, at the 3′ end, with the polyadenylation signal for octopine synthetase of the T-DNA, and is under the control of the B33 promoter.

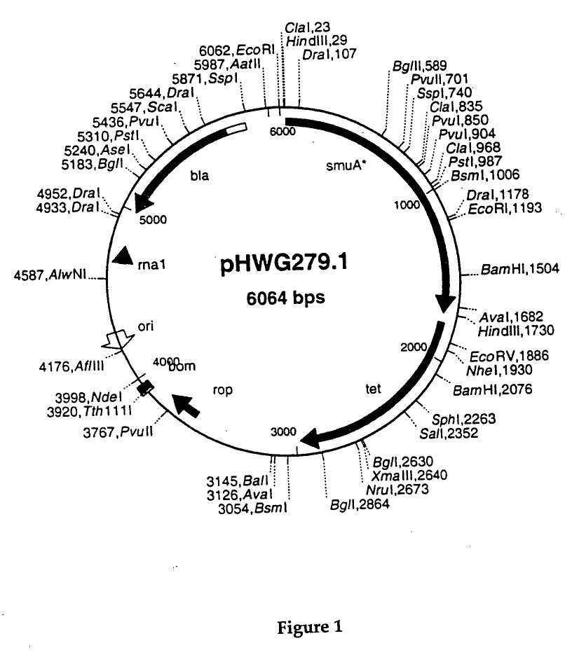

A HindIII fragment of about 1.7 kb (containing the sucrose isomerase coding sequence) of the plasmid pHWG279.1 shown in FIG. 1 (which was kindly made available by Prof. Mattes, University of Stuttgart), containing the native gene for sucrose isomerase from Protaminobacter rubrum in the vector pBR322 (Bolivar et al., Gene, 2 (2) (1977), 95-113; Peden, Gene 22 (2-3) (1983), 277-280), was cloned in the vector pBluescriptSK (Stratagene, Heidelberg), producing a plasmid designated as pSK279.1. The signal sequence of the patatine gene was amplified in the PCR process for “in frame” cloning of a vacuolar transit peptide of the patatine gene (297 bp, Rosahl et al., 1986). After the ends were removed with the restriction enzymes APpal and SalI, it was cloned in pBluescriptSK, producing the plasmid pSK297. The plasmid pSK297 was cleaved with the restriction enzyme SalI. The protruding ends were converted into blunt ends and then ligated with the 1.7 kb fragment of the plasmid pSK279.1, the ends of which had previously been converted to blunt ends. The plasmid obtained was designated as UL5-19. As a check, the transition region between the signal sequence and the nucleotide sequence coding for sucrose isomerase was sequenced to make sure that the transition was correct. It was found that, although the transition was correct, the nucleotide sequence of the plasmid pHWG279.1 coding for sucrose isomerase contained several sequence errors, including a stop codon. Therefor the HindIII fragment was replaced by an error-free HindIII fragment coding for sucrose isomerase. The pHWG432.3 plasmid obtained was transformed in Escherichia coli DH5alpha and tested for enzymatic activity. The cDNA fused with the signal sequence was isolated by cleaving the plasmid pHWG432.3 with XbaI and trimming the protruding ends, followed by cleaving with Asp718. The 2.0 kB fragment obtained by that procedure was cloned in the binary vector pBinB33-Hyg, which had been cleaved with SalI, so that the protruding ends were trimmed, and Asp718, so that directed cloning was possible. The vector obtained was designated as UL8-19. It was used to transform the Agrobacterium tumefaciens strain pGV2260 (Deblaere et al., Nucl. Acids Res., 13 (1985), 4777-4788) by electroporation. The transformed agrobacteria were used to transform the AGPase-antisense line 93 of the potato (“sucrose potato”; Müller-Röbber et al., 1990) and the potato wild type variety Desiree. The transformed plants were designated 086BK and 096BK, respectively.

### EXAMPLE 2

### Production of Vectors Containing a Nucleotide Sequence Coding for Sorbitol Dehydrogenase

A series of constructs was produced. Each contained the nucleotide sequence coding for sorbitol dehydrogenase from Gluconobacter suboxidans and the polyadenylation signal from T-DNA octopine synthase in a binary vector containing a promoter expressible in plant cells. In this example, too, the coding nucleotide sequence was either fused with the signal sequence of the patatine gene in order to attain vacuolar localization of the gene product, or used without the vacuolar target sequence to express the gene product in the cell cytosol. The CaMV 35 S-promoter or the B33 promoter from the B33 gene of the potato were used as promoters. The binary vector pBinB33-Hyg, which already contains the B33 promoter and the polyadenylation signal, was used for the potato. The binary vector pGA492 was used for the sugar beet. Agrobacteria were transformed with the plasmids obtained. The transformed agrobacteria were used to transform either potato or sugar beet. The construction of the plasmid U120/19 is described below. In that process the sequence coding for the sorbitol dehydrogenase is fused “in frame” at the 5′ end with the signal peptide of the patatine gene and, at the 3′ end, with the polyadenylation signal of the T-DNA and is under the control of the B33 promoter.

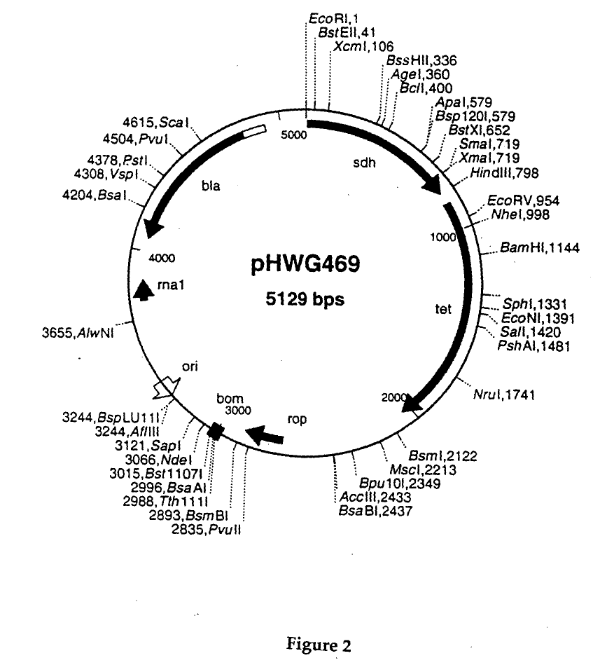

The vector pSK297 (pBluescript with 297 bp of the vacuolar target sequence of the patatine gene) was cleaved with the restriction enzyme EcoRV. Prof. Mattes, University of Stuttgart, kindly made available the plasmid pHWG469 (see FIG. 2). It contains the native gene for sorbitol dehydrogenase from Gluconobacter suboxidans in the vector pBR322 (Bolivar et al., Gene, 2 (2) (1977), 95-113; Peden, Gene 22 (2-3) (1983) 277-280). A fragment coding for sorbitol dehydrogenase, from Gluconobacter suboxidans, was cut out of it by cleavage with the restriction enzymes EcoRV and HindIII. After the protruding ends were converted to blunt ends, it was cloned after the vacuolar target sequence, producing a general reading frame. The reading frame was checked by sequencing at the site of fusion. A fragment containing 1145 bp which codes for the fused protein was cut out of the plasmid UL19/19 obtained in that way by cleaving with the restriction enzymes Asp718 and BamHI. This fragment was cloned in the binary vector pBinB33 (Becker, NAR, 18 (1990), 203). The resulting plasmid, UL20/19, was used to transform the Agrobacterium tumefaciens strain pGV2260. The transformed strains were used to transform lines 21 and 33 of the potato line 096BK. That produced the transgenic plants, 158BK and 159BK, respectively.

### Example 3

### Production of a Binary Vector Containing a Nucleotide Sequence Coding for Mannitol Dehydrogenase

A series of constructs was prepared. Each one contained, in a binary vector, the nucleotide sequence from Pseudomonas fluorescens DSM 50106 coding for mannitol dehydrogenase (Brünker et al., Biochimica et Biophysica Acta, 1351 (1997), 157-167) together with a plant-specific promoter and the polyadenylation signal from T-DNA octopine synthase. In this example, also, the coding nucleotide sequence was either fused with the signal sequence of the patatine gene to achieve vacuolar localization of the gene product, or used without the vacuolar target sequence to express the gene product in the cell cytosol. The CaMV 35 S promoter or the B33 promoter of the B33 gene of the potato were used as promoters. In the case of the potato, the binary vector pBinB33-Hyg, which already contains the B33 promoter and the polyadenylation signal, was used. The binary vector pGA492 was used for the sugar beet. Agrobacteria were transformed with the plasmids obtained. The transformed agrobacteria were used to transform either the potato or the sugar beet.

### Example 4

### Transformation of Agrobacterium tumefaciens

The DNA transfer into the agrobacteria was accomplished by means of direct transformation using the procedure of Höfgen and Willmitzer (Nucl. Acids Res., 16 (1988), 9877). The agrobacteria transformed by the plasmid DNA were isolated by the procedure of Birnboim and Doly (Nucl. Acids Res., 7 (1979), 1513-1523) and were analyzed by gel electrophoresis after suitable restriction cleavage.

### EXAMPLE 5

### Transformation of the Potato

The plant transformation was accomplished by gene transfer using the process described by Dietze et al., Gentransfer to Plants (1995), 24-29, mediated by Agrobacterium tumefaciens (strain pGV2260 in C58C1; Deblaere et al., Nucl. Acids Res., 13 (1985), 4777-4788. The transformed plants were selected on media containing either kanamycin or hygromycin.

### EXAMPLE 6

### Induction of Regenerable Calluses from Leaves of the Sugar Beet

About one month after germination of sugar beet seeds in the greenhouse, experiments on induction of calluses were done by the procedure described by Saunders et al. (Saunders, J. W. and Doley, W. P., J. Plant. Physiol., 124 (1986) 473-479). A young leaf, three to five centimeters long, was removed from each plant, disinfected, washed three times with sterile water, and dried on sterile filter paper. Then each leaf was cut into segments of about 0.25 cm², and the explants obtained in this way were cultivated on MSB1 medium in Petri dishes. After the dishes had been sealed air-tight with a plastic film, they were incubated for thirty days in the dark at 30° C. Then they were transferred to culture chambers. White brittle calluses appeared on or under the leaf explants four to ten weeks after the beginning of the culture.

### EXAMPLE 7

Production of Cell Suspensions from Calluses Induced on the Sugar Beet

The calluses were removed four to six weeks after they appeared, and were cultivated in 100 ml liquid MSB1 medium in 250 ml Erlenmeyer flasks sealed with film. The Erlenmeyer flasks were shaken on a rotary shaker at about 200 rpm. A cell suspension was obtained after about two to three weeks.

### EXAMPLE 8

### Transformation of Cell Suspensions and Young Calluses of the Sugar Beet Cell Suspensions

The transformation was done with cell suspensions after cultivation for about three weeks. Ten milliliters of fresh MSB1 medium was added to 10 ml of the suspension medium. The suspension, diluted in that manner, was distributed over four Petri dishes.

Fifty microliter portions were taken from stock cultures of Agrobacterium tumefaciens strains which had been transformed with the binary vectors produced. They were cultured in 2 ml LB medium containing rifampicin and tetracycline. The cultures were stirred at 200 rpm for two days at 30° C. This strain was transferred to fresh medium and cultured over night under the conditions described above.

The plant cells were infected by adding 50 μl of an Agrobacterium tumefaciens strain to each of the Petri dishes containing the corresponding beet cells. The beet cells and the bacteria were cultured in the dark in a culture chamber for three days. Then the bacteria were removed from the plant cells by washing first with MSB1 and 600 mg/liter cefotaxim and then with MSB1 plus 300 mg/liter cefotaxim. The beet cells washed in that manner were cultivated in Petri dishes on a sheet of sterile Whatman paper lying on an MSB1 medium containing kanamycin plus 300 mg/liter cefotaxim. The dishes were sealed air-tight with plastic film and incubated in the culture chamber for fifteen days. Three to eight weeks later, white calluses appeared on a layer of dead cells.

### Dispersion of New Induced Calluses

Young calluses, freshly induced from leaf explants, were transformed. The calluses used had appeared on leaves after two to six weeks. Those calluses were dispersed in liquid MSB1 medium in sterile plastic tubes and subjected to the same transformation process as the cell suspensions.

### EXAMPLE 9

### Regeneration of Sugar Beet Plants from Transformed Calluses

After the transformed calluses had been cultivated initially for one month on MSB1 and cefotaxim or on MSB1 and cefotaxim and kanamycin, cultivation of the transformed calluses was continued on MSB1 medium.

After a certain period, sprouts and/or embryos developed from certain calluses. As soon as the sprouts had started to develop leaves, they were rooted in MS medium containing 1 mg/liter of naphthaleneacetic acid. Roots appeared after two to six weeks. After roots developed, the plants were acclimatized in humus soil in the greenhouse. Complete plants had developed after another three months.

### Example 10

### Demonstration of the Mannitol Dehydrogenase, Sorbitol Dehydrogenase and Sucrose Isomerase Genes Produced in the Transformed Tissue

Transformed plants were preselected on media containing kanamycin or hygromycin. To detect the transgenes, genomic DNA was first isolated from the corresponding tissues (potato tubers or sugar beet storage roots). Thirty nanogram portions of genomic DNA were used as templates for the polymerase chain reaction (PCR; Saki et al., Science, 239 (1988), 487-491). The primers were gene-specific probes from the 5′ and 3′ regions of sucrose isomerase from Protaminobacter rubrum, of sorbitol dehydrogenase from Gluconobacter suboxidans, and of mannitol dehydrogenase from Pseudomonas fluorescens. Each reaction was carried out in a solution with a total volume of 50 μl containing 1 μM 3′ and 5′ primers, 0.2 mM dNTPs, 1.5 mM MgCl₂, 50 mM KCl and 20 mM Tris-HCl, pH 8.4, with 1 unit of Tag polymerase (Gibco-BRL). The PCR solutions were each subjected to 40 cycles with 1 minute denaturation at 95° C., 1 minute of primer attachment at 65° C. and 2.5 minutes of synthesis at 72° C., with the entire reaction terminated by a final ten-minute synthesis for chain completion. The reaction products were analyzed by gel electrophoresis. The particular PCR products corresponding to the transgenes were determined by the fragment size. After subcloning and partial sequencing of the PCR products, the transgenes could be identified unambiguously.

### Example 11

### Demonstration of the Sucrose Isomerase Activity in Transformed Tissue

Sucrose isomerase activity was demonstrated in transformed potato tubers as follows. Potato tubers from transgenic potato plants, and tubers from the wild type variety Desiree used as the control were ground up. Portions of 2 to 5 grams of the ground material were homogenized in an Omni-Mixer after addition of 50 ml boiling water, and then heated for 15 minutes in a water bath at 95° C. Isomaltulose was detected with the HPAEC procedure after centrifugation and dilution of the supernatant. Table 1 shows the results obtained.

TABLE 1
Detection of isomaltulose in transgenic potato plants

| | g isomaltulose/kg fresh weight |
|---|---|
| Desiree 1 | 0.0 |
| Desiree 2 | 0.0 |
| Desiree 3 | 0.0 |
| Transgenic sample 1 | 23.6 |
| Transgenic sample 2 | 14.0 |
| Transgenic sample 3 | 44.8 |
| Transgenic sample 4 | 31.4 |
| Transgenic sample 5 | 38.5 |

1. Transgenic plant which can in at least one of its cells produce isomaltulose from the sucrose formed in the plant and can produce 6-O-α-D-glucopyranosyl-D-sorbitol (1,6-GPS) and/or O-α-D-glucopyranosyl-D-mannitol from the isomaltulose produced,
characterized in that
the plant contains, in the at least one cell, a stable integrated nucleotide sequence which is expressible in it, which codes for the activity of a sucrose isomerase to isomerize sucrose to isomaltulose, and at least one stable integrated and expressible nucleotide sequence, selected from the group comprising a nucleotide sequence which codes for the activity of a sorbitol dehydrogenase to reduce isomaltulose to 1,6-GPG and a nucleotide sequence which codes for the activity of a mannitol dehydrogenase to reduce isomaltulose to 1,1-GPM.

2. Transgenic plant which contains in at least one of its cells a stable integrated and expressible nucleotide sequence which codes for the activity of a sorbitol dehydrogenase to reduce isomaltulose to 1,6-GPS.

3. Transgenic plant which contains in at least one of its cells a stable integrated and expressible nucleotide sequence which codes for the activity of a mannitol dehydrogenase to reduce isomaltulose to 1,1-GPM.

4. Transgenic plant according to one of claims 1 to 3,
characterized in that
it is a potato.

5. Transgenic plant according to one of claims 1 to 3,
characterized in that
it is a sugar beet.

6. Transgenic plant according to one of claims 1 to 5,
characterized in that
the nucleotide sequence is contained in a plant vector.

7. Transgenic plant according to claim 6 in which the coding nucleotide sequence is a cDNA or a genomic DNA sequence.

8. Transgenic plant according to claim 7, in which the nucleotide sequence coding for sucrose isomerase can be obtained from a microorganism, especially from a microorganism of the genus Protaminobacter, Erwinia, Serratia, Leuconostoc, Pseudomonas, Agrobacterium or Klebsiella, the nucleotide sequence coding for sorbitol dehydrogenase can be obtained from a microorganism, especially from a microorganism of the genus Gluconobacter, and the nucleotide sequence coding for mannitol dehydrogenase can be obtained from a microorganism, especially from a microorganism of the genus Pseudomonas.

9. Transgenic plant according to one of claims 1 to 8, in which the coding nucleotide sequence is under the functional control of at least one regulatory element which assures the transcription in plant cells.

10. Transgenic plant according to claim 9, in which the at least one regulatory element is a promoter, especially a plant-specific promoter.

11. Transgenic plant according to claim 10, in which the promoter is a tissue-specific or organ-specific promoter, preferably a storage-organ-specific promoter.

12. Transgenic plant according to one of claims 1 to 11, in which the coding nucleotide sequence in the reading frame is either fused with a signal sequence which codes for a signal peptide which assures transport of the protein with the activity of a sucrose isomerase, the protein with the activity of a sorbitol dehydrogenase, or the protein with the activity of a mannitol dehydrogenase to a particular cell compartment or a particular cell organelle, or is not fused with a signal sequence, so that the protein with the activity of a sucrose isomerase, the protein with the activity of a sorbitol dehydrogenase, or the protein with the activity of a mannitol dehydrogenase is localized in the cytosol.

13. Transgenic plant according to one of claims 1 to 12, in which the coding nucleotide sequence is functionally linked with termination and/or polyadenylation signals.

14. Propagation and/or harvest material from a plant according to one of claims 1 to 13.

15. Process for producing a transgenic plant according to one of claims 1 to 3, comprising
a) transformation of one or more plant cells with one or more nucleotide sequence(s) selected from the group consisting of a nucleotide sequence coding for the activity of a sucrose isomerase, a nucleotide sequence coding for the activity of a sorbitol dehydrogenase, and a nucleotide sequence coding for the activity of a mannitol dehydrogenase.
b) integration of the nucleotide sequence(s) into the genome(s) of the transformed cell(s), and
c) regeneration of plants which produce the sorbitol dehydrogenase, mannitol dehydrogenase and/or sucrose isomerase.

16. Process according to claim 15, in which the transformation is a cotransformation of the individual nucleotide sequences used.

17. Process according to claim 16, in which the cells to be transformed are transgenic cells which contain at least one stable integrated nucleotide sequence, selected from the group consisting of a nucleotide sequence coding for the activity of a sucrose isomerase, a nucleotide sequence coding for the activity of a sorbitol dehydrogenase, and a nucleotide sequence coding for the activity of a mannitol dehydrogenase.

18. Process according to one of claims 15 to 17,
characterized in that
the transformed nucleotide sequence(s) is/are contained in a plant vector.

19. Process according to claim 18, in which the coding nucleotide sequence(s) is/are a cDNA or a genomic DNA sequence.

20. Process according to claim 19, in which the nucleotide sequence coding for sucrose isomerase can be obtained from a microorganism, especially from a microorganism of the genus Protaminobacter, Erwinia, Serratia, Leuconostoc, Pseudomonas, Agrobacterium or Klebsiella, the nucleotide sequence coding for sorbitol dehydrogenase can be obtained from a microorganism, especially from a microorganism of the genus Gluconobacter, and the nucleotide sequence coding for mannitol dehydrogenase can be obtained from a microorganism, especially from a microorganism of the genus Pseudomonas.

21. Process according to one of claims 15 to 20, in which each of the coding nucleotide sequence(s) is/are under the functional control of a regulatory element which assures transcription in plant cells.

22. Process according to claim 21, in which the at least one regulatory element is a promoter, especially a plant-specific promoter.

23. Process according to claim 22, in which the promoter is a tissue-specific or organ-specific promoter, preferably a storage-organ-specific promoter.

24. Process according to one of claims 15 to 23, in which the coding nucleotide sequence(s) is/are either fused with a signal sequence in the reading frame which codes for a signal peptide which assures transport of the coded protein to a specific cell compartment or a specific cell organelle, or is/are not fused with a signal sequence, so that the coded protein is localized in the cytosol.

25. Process according to one of claims 15 to 24, in which the coding nucleotide sequence(s) is/are functionally linked with termination signals and/or polyadenylation signals.
